(19) United States
(12) Patent Application Publication
(10) Pub. No.: US 2013/0172700 A1
(43) Pub. Date: Jul. 4, 2013

(54) OPTICAL DETECTION METHOD

(71) Applicants: Yung-Sung Lan, Kaohsiung City (TW); Chir-Weei Chang, Taoyuan County (TW)

(72) Inventors: Yung-Sung Lan, Kaohsiung City (TW); Chir-Weei Chang, Taoyuan County (TW)

(73) Assignee: INDUSTRIAL TECHNOLOGY RESEARCH INSTITUTE, Hsinchu (TW)

(21) Appl. No.: 13/426,559

(22) Filed: Mar. 21, 2012

(30) Foreign Application Priority Data
Dec. 30, 2011 (TW) 100149871

(51) Int. Cl. A61B 3/10

(52) U.S. Cl. 600/318

(57) ABSTRACT

An method for detection of β-amyloid in aqueous humor, lens, and retina of eye or the deposit of the combination of αβ-crystallin and β-amyloid in lens is provided. A light is emitted to a testing area in the eye. The light frequency is selected according to an absorption spectrum of the test substance, and the frequency is equal or close to a resonant excitation frequency of one of the electronic molecular energy levels of the substance, so as to excite the substance to generate resonance-enhanced Raman effect or pre-resonance Raman effect to form a detection spectrum. The concentration of the substance could be estimated by a peak intensity of the detection spectrum.

### CROSS-REFERENCE TO RELATED APPLICATION

This application claims the priority benefit of Taiwan application serial no. 100149871, filed on Dec. 30, 2011. The entirety of the above-mentioned patent application is hereby incorporated by reference herein and made a part of this specification.

### TECHNICAL FIELD

The technical field relates to a detection method, and more particularly to an optical detection method.

### BACKGROUND

There are approximately 25 million people in the world with dementia, and the number doubles every 20 years. According to data from the United States Census Bureau, the percentage of the population age 65 years and over will drastically increase. Since 5-6% of the population is afflicted with Alzheimer's disease or related forms of dementia, this represents approximately 4 million Americans have Alzheimer's disease. As the patients age and conditions deteriorate, 5-10% of the population over age 65 and over half of the population over age 85 suffer from Alzheimer's disease. Consequently, the burdens placed on the caretakers and to society are rising day by day, with about $100 million US dollars spent yearly on the patients. It is predicted that by 2050, there will be 14 million Americans with Alzheimer's disease. Moreover, Alzheimer's disease will become the 4^{th} leading cause of death, accounting for 100 thousand deaths per year. In 2005, Taiwan has close to 140 thousand patients with dementia. It is estimated that by the year 2050, 660 thousand people will have dementia. Furthermore, among all the sufferers of dementia, approximately 55% (363 thousand people) will be Alzheimer's patients. The Alzheimer's disease is a sustained impediment to nerve functions and one of the most common causes of dementia. Impediments to higher cortical functions include emotional control and societal behaviors. The impediments for societal behaviors may include: memory loss, impaired thinking, progressive reduction of learning capacity, loss of language, and altered judgment. These symptoms place extremely heavy burdens on the families of the patients and contribute to the increase in societal costs.

Currently, an accurate diagnosis of Alzheimer's disease is difficult to obtain other than performing an autopsy on the brain of the deceased patient in order to discover the symptoms of the disease. Clinically, various combinations of tests and measurements are performed to increase the accuracy of the diagnosis. The most commonly adopted standard of diagnosis is The Diagnosis and Statistical Manual of Mental Disorders published by the American Psychiatric Association. In addition, the contents of the disease assessment further include the medical history, the psychiatric evaluations, and the psychological, physiological, and nerve function examinations. With old conventional medical techniques, diagnoses and treatments are possible only when the disease has developed or during the late stages of the disease.

In 2006, Neuroptix Corp. developed an optical apparatus for early diagnosis of Alzheimer's disease capable of preventing continued deterioration through early diagnosis. The Neuroptix optical system, known as the QEL 2400, performs the early diagnosis of Alzheimer's disease by first applying an eye drop in an eye containing a fluorescent reagent (the fluorescent reagent combines with β-amyloid and has specificity thereto). Next, when an infrared laser scans the eye lens, the fluorescent reagent emits fluorescent light, and accordingly the concentration of β-amyloid in the lens can be determined, thereby achieving the early diagnosis of Alzheimer's disease.

In 2010, a research team from the newly acquired Avid Radiopharmaceuticals Inc. of Eli Lilly and Co. indicated that, in advanced stage clinical trials, their imaging agent can accurately detect β-amyloid correlated to Alzheimer's disease. In another research study by Dr. Kristine Yaffe at the Veterans Affairs Medical Center, certain types of blood test methods can predict whether a patient is at risk of dementia several years before symptoms start to appear for the patient. Avid Radiopharmaceuticals has taken the lead ahead of General Electric Co. and Bayer AG to sell imaging agents capable of detecting Alzheimer's disease before the sufferer's death. Florbetapir F18, a radioactive drug developed by Avid Radiopharmaceuticals, is used in conjunction with positron emission tomography (PET) scans.

In 2011, Stanford University announced an innovative neuron imaging technique in the brain, capable of capturing the neural activities in the brain for several months. This state-of-the-art imaging method will help medical practitioners to understand and treat nerve related diseases, such as Alzheimer's, dementia, and brain cancer.

Three important tools in medical imaging include the computerized tomography (CT) scanner, the PET scanner, and the magnetic resonance imaging scanner, and each scanner has its own advantages. The CT and MRI scanners scan for structural anomalies, whereas the PET scanner detects functional problems. Moreover, the CT scan can be used to view the degree of brain shrinkage. The CT scanner is a diagnostic tool combining X-ray and computer computations. The X-ray source is illuminated at different angles of the body, and the computer processes the data into cross-sectional images of the body. When the sulci on the surface of the brain expands in width, the ventricles (space in the brain filled with cerebrospinal fluid) are enlarged, thereby forming some of the characteristics of Alzheimer's disease.

Conventionally, Alzheimer's disease is typically detected by using imaging agents, fluorescent agents, or radioactive drugs in conjunction with expensive testing instruments. These methods require labeling with the imaging agents, fluorescent agents, or the radioactive drugs, and the testing instruments are expensive. Faced with so many Alzheimer's patients, it is often extremely difficult to detect Alzheimer's disease early.

Research has indicated that Alzheimer's disease has two major symptoms, the first being beta amyloid plaques which many researchers believe to be amyloid β-protein (Aβ) deposited in the neocortex and the hippocampus causing neuron damage. The protein deposit of the insoluble beta amyloid plaques is a type of protein cut by enzymes from a larger protein (amyloid precursor protein (APP)). The second symptom is neurofibrillary tangles formed by the clumping of microtubule-associated protein tau. At the same time, neurofibrillary tangle proteins can be found in the neurons, marking another characteristic of neurons in Alzheimer's patients.

The fundamental cause behind the toxicity of Alzheimer's disease is the amyloid β-protein. During analysis, other microglia or reactive astrocytes surround the neuritic plaques. A major component of the neuritic plaques is the amyloid β-protein. In particular, the deposits of the neuritic plagues have different lengths, and a large part of the initially discovered proteins are proteins of 40 amino acids, referred to as the amyloid β-protein (1-40) (Abeta₁₋₄₀), with around 90% of the volume. The rest of the Abeta are proteins of 42 amino acids (referred to as the amyloid β-protein (1-42) (Abeta₁₋₄₂). The addition of two amino acids results in the protein's increased hydrophobic property, and therefore the amyloid β-protein is even more likely to be deposited and accumulated around the cells. The same protein molecules are then attracted, thereby the seeding core slowly grows and completes the structure of the entire neuritic plaque. It is commonly believed that senile plaques of the insoluble form are formed by the self-aggregation of amyloid β-protein (β-amyloid or Aβ) into fibrils of several micrometers in length after undergoing a complex chain of reactions, and the amyloid fibrils combining to form the senile plaques. The neurons surrounding the insoluble senile plaques undergo the neuro degeneration process and experience cell death.

Moreover, research has indicated that β-amyloid is deposited in the brain and the aqueous humor, lens, and retina of the eye of the Alzheimer's patient. In addition, after αβ-crystallin combines with β-amyloid in the eye lens of the Alzheimer's patient, a large absorption spectrum near 450 nm can be detected.

### SUMMARY

The disclosure provides an optical detection method, capable of detecting a concentration of β-amyloid in a human eye in a non-invasive and label-free test to provide an early diagnosis of Alzheimer's disease.

The disclosure provides an optical detection method, capable of detecting a concentration of a deposit of a combination of αβ-crystallin and β-amyloidin in a lens of a human eye in a non-invasive and label-free test to provide an early diagnosis of Alzheimer's disease.

According to an embodiment, a method for detecting a concentration of a substance in an eye is provided. The method includes the following steps. First, β-amyloid (Aβ) is selected as the substance. A light is emitted to a testing area in the eye, in which a frequency of the light is selected according to an absorption spectrum of the selected substance, and the frequency is equal or close to a resonant excitation frequency of one of the electronic molecular energy levels of the test substance, so as to excite the test substance to generate resonance-enhanced Raman effect or pre-resonance Raman effect to form a detection spectrum. Next, the detection spectrum is received and the concentration of the test substance is estimated according to a peak intensity of the detection spectrum.

According to an embodiment, a method for detecting a concentration of a deposit of a combination of αβ-crystallin and β-amyloid in a lens of an eye is provided. The method includes the following steps. First, a light is emitted to the lens, in which a frequency of the light is selected according to an absorption spectrum of the test substance, and the frequency of the light is equal or close to a resonant excitation frequency of one of the electronic molecular energy levels of the deposit of the combination of αβ-crystallin and β-amyloid, so as to excite the deposit of the combination of αβ-crystallin and β-amyloid to generate resonance-enhanced Raman effect or pre-resonance Raman effect to form a detection spectrum. Next, the detection spectrum is received and the concentration of the deposit of the combination of αβ-crystallin and β-amyloid is estimated according to a peak intensity of the detection spectrum, so as to obtain a concentration of β-amyloid for determining the severity of Alzheimer's disease.

In summary, due to the light excitation of β-amyloid or the deposit of the combination of αβ-crystallin and β-amyloid to generate resonance-enhanced Raman effect or pre-resonance Raman effect, the optical method according to some embodiments of the disclosure can enhance the measured signal of the substance with the resonance-enhanced Raman effect. Accordingly, the concentration of β-amyloid can be accurately estimated, thereby enabling the early diagnosis of Alzheimer's disease.

Several exemplary embodiments accompanied with figures are described in detail below to further describe the disclosure in details.

### BRIEF DESCRIPTION OF THE DRAWINGS

The accompanying drawings constituting a part of this specification are incorporated herein to provide a further understanding of the disclosure. Here, the drawings illustrate embodiments of the disclosure and, together with the description, serve to explain the principles of the disclosure.

FIG. 1 is a flowchart of an optical detection method according to an embodiment of the disclosure.

FIG. 2A is a Raman spectrum diagram of β-amyloid (1-40) measured by a 632.8 nm incident light.

FIG. 2B is a Raman spectrum diagram of β-amyloid (1-42) measured by an incident light of 632.8 nm.

FIG. 3A is a Raman spectrum diagram of β-amyloid (1-40) measured by a 514.5 nm incident light.

FIG. 3B is a Raman spectrum diagram of β-amyloid (1-42) measured by an incident light of 514.5 nm.

FIG. 4A is a schematic view of an optical path when using the optical detection method depicted in FIG. 1 to perform detection in an aqueous humor.

FIG. 4B is a schematic view of an optical path when using the optical detection method depicted in FIG. 1 to perform detection in a lens.

FIG. 4C is a schematic view of an optical path when using the optical detection method depicted in FIG. 1 to perform detection in a retina.

### DETAILED DESCRIPTION OF DISCLOSED EMBODIMENTS

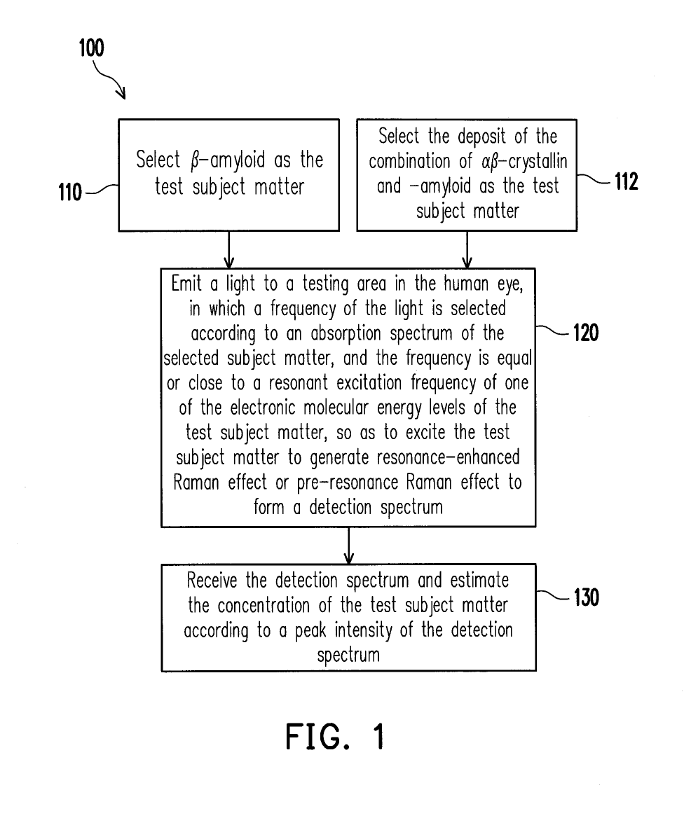

FIG. 1 is a flowchart of an optical detection method according to an embodiment of the disclosure. Referring to FIG. 1, the optical detection method of the present embodiment is for detecting a concentration of a substance in a human eye. The substance generates a Raman spectrum after being illuminated, in which the substance includes the resonant modes of a plurality of electronic molecular energy levels.

The Raman spectrum may reflect the molecular structural properties. Moreover, when the frequency of the incident light is equal or extremely close to the transitional frequency of the molecular energy levels of the substance being detected, the intensity of the scattered light drastically increases and may resonant with the particular substance, thereby enhancing the signal strength by several orders of magnitude. Therefore, the optical detection method can be used to measure trace concentrations, and to lower the power of the incident light so as to prevent eye damage.

An optical detection method 100 of the present embodiment includes the following steps. In a Step S110, β-amyloid (Aβ) is selected as the substance. β-amyloid may be β-amyloid (1-40) or β-amyloid (1-42). Since the deposit concentration of β-amyloid is considerably correlated with Alzheimer's disease, therefore, by obtaining the Raman spectrum of β-amyloid, the degree of the Alzheimer's disease can be determined.

Thereafter, an absorption spectrum of the selected substance is measured, in which the wavelength with the largest absorption has a resonance effect. In a Step S120, a light is emitted to a testing area, in which a frequency of the light is selected according to the absorption spectrum, and the frequency is equal or close to a resonant excitation frequency of one of the electronic molecular energy levels of the test substance, so as to excite the test substance to generate resonance-enhanced Raman effect or pre-resonance Raman effect to form a detection spectrum. In the present embodiment, the testing area may be an aqueous humor, a lens, or a retina, and the detection spectrum is a Raman spectrum. The detected substance of the embodiment is β-amyloid, and for example the detection light with a wavelength range from 300 nm to 330 nm (e.g. 315 nm) can be adopted to be emitted to the testing area, and the detection spectrum can be obtained. However, the wavelength of the detection light is not limited to the range from 300 nm to 330 nm and may be varied with actual detection demands or location of detected object. In other words, the light with a wavelength range greater than 330 nm or less than 300 nm can be selected to 330 nm be emitted to the testing area, to obtain the detection spectrum.

Next, in a Step S130, the detection spectrum is received and the concentration of the test substance is estimated according to a peak intensity of the detection spectrum. Deposits of β-amyloid are found in the aqueous humor, lens, or retina of the eye of the Alzheimer's patient. Therefore, the concentration of β-amyloid can be respectively measured for the aqueous humor, lens, or retina. Moreover, the accuracy of the diagnosis can be increased by evaluating the measurement results together.

In addition, the eye lens of the Alzheimer's patient has deposits of the combination of αβ-crystallin and β-amyloid. Thus, in a Step S112, the test substance can be selected as the deposits of the combination of αβ-crystallin and β-amyloid. Then, by using light with a wavelength range of 440 nm to 460 nm to perform Steps 120 and 130, the concentration of the deposits of the αβ-crystallin and β-amyloid combination can be obtained to determine the severity of Alzheimer's disease. It should be appreciated that, if another substance is deposited in the human eye according to a symptom of Alzheimer's disease, this substance can be selected as the test substance. By using the optical detection method to detect the concentration of the test substance, the severity of Alzheimer's disease can be estimated.

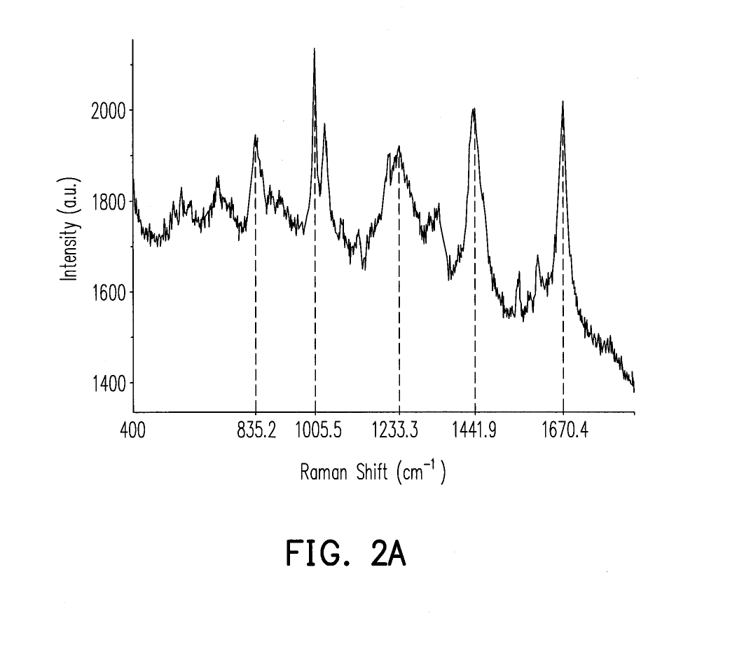

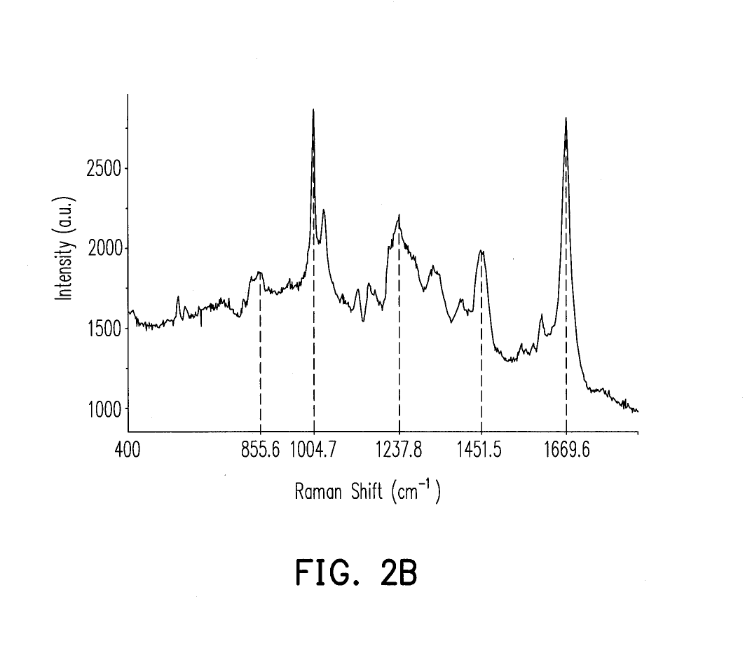

FIG. 2A is a Raman spectrum diagram of β-amyloid (1-40) measured by an incident light with wavelength of 632.8 nm. FIG. 2B is a Raman spectrum diagram of β-amyloid (1-42) measured by an incident light of 632.8 nm. Referring to FIGS. 2A and 2B, the Raman shifts of the β-amyloid (1-40) and β-amyloid (1-42) are approximately between 400 cm⁻¹ to 2000 cm⁻¹. As shown in FIG. 2A, the Raman shifts of β-amyloid (1-40) are approximately located at 835.2, 1005.5, 1233.3, 1441.9, and 1670.4 cm⁻¹. Moreover, as shown in FIG. 2B, the Raman shifts of β-amyloid (1-42) are approximately located at 855.6, 1004.7, 1237.8, 1451.5, and 1669.6 cm⁻¹.

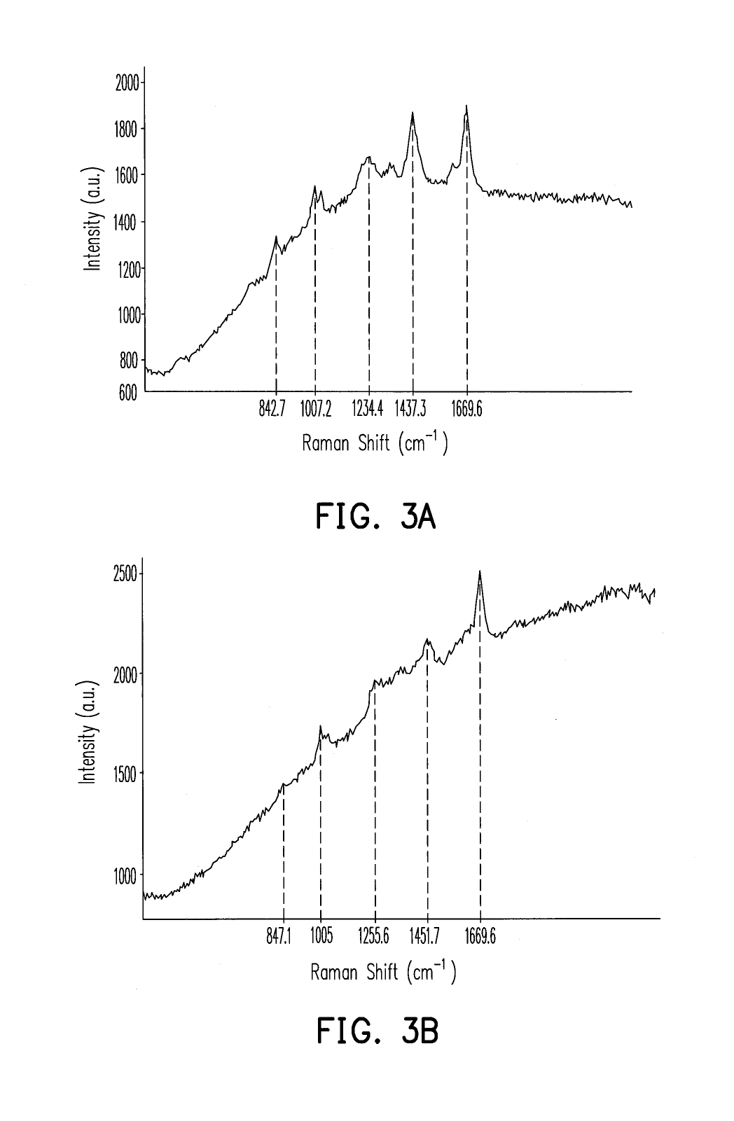

FIG. 3A is a Raman spectrum diagram of β-amyloid (1-40) measured by an incident light with wavelength of 514.5 nm. FIG. 3B is a Raman spectrum diagram of β-amyloid (1-42) measured by an incident light of 514.5 nm. As shown in FIG. 3A, the Raman shifts of β-amyloid (1-40) are approximately located at 842.7, 1007.2, 1234.4, 1457.3, and 1669.6 cm⁻¹. Moreover, as shown in FIG. 3B, the Raman shifts of β-amyloid (1-42) are approximately located at 847.1, 1005.0, 1255.6, 1451.7, and 1669.6 cm⁻¹. The concentrations of β-amyloid (1-40) and β-amyloid (1-42) can be estimated according to the peak intensities of the Raman spectrum.

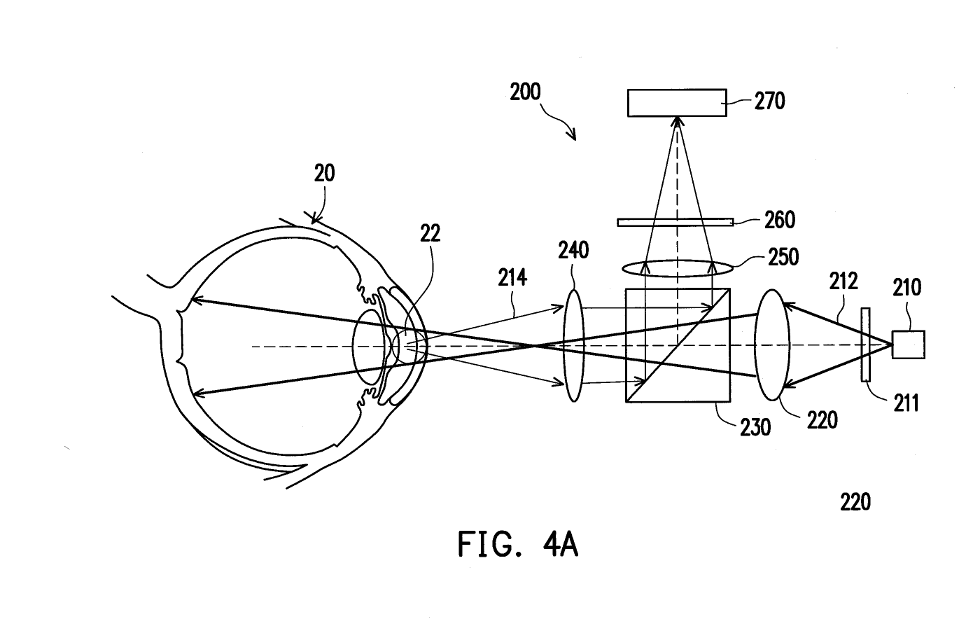

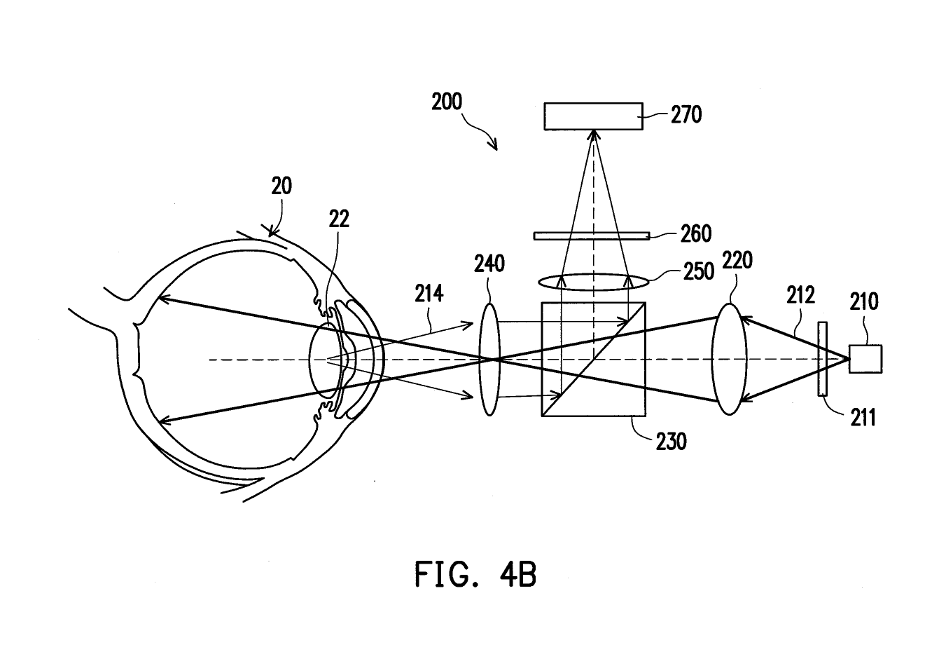

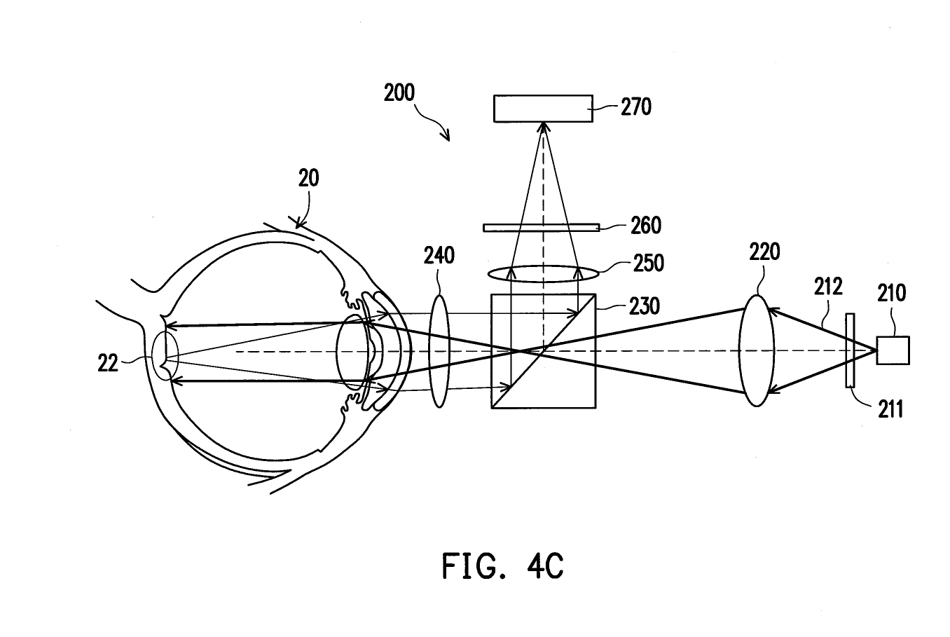

FIG. 4A is a schematic view of an optical path when using the optical detection method depicted in FIG. 1 to perform detection in an aqueous humor. FIG. 4B is a schematic view of an optical path when using the optical detection method depicted in FIG. 1 to perform detection in the lens. FIG. 4C is a schematic view of an optical path when using the optical detection method depicted in FIG. 1 to perform detection in the retina.

When performing the Steps 120 and 130 in FIG. 1, the detection spectrum can be obtained by referring to the optical detection devices 200 and the optical paths from FIGS. 4A to 4C. As shown in FIGS. 4A to 4C, a light 212 is emitted from a light source 210 and is focused in front of an eye 20 by passing through a first lens 220. In the present embodiment, the light source 210 is a light emitting diode (LED), although in other embodiments, the light source may be a laser diode. The light source 210 passes through a pinhole and becomes a point light source. The source light then passes through a first filter 211 to ensure the light 212 close to a resonant wavelength of the test substance enters the eye. The light 212 is transmitted to a testing area 22 of the eye 20 by passing through a beam splitter 230. In FIG. 4A, the testing area 22 is the aqueous humor. In FIG. 4B, the testing area 22 is the lens. In FIG. 4C, the testing area 22 is the retina.

A second lens 240 is disposed between the testing area 22 and the beam splitter 230, and a distance from the testing area 22 to the second lens 240 is substantially equal to a focal length of the second lens 240. After passing through the testing area 22 (located at a focal point of the second lens 240), a detection light 214 is generated. The detection light 214 becomes a parallel light after passing through the second lens 240 and is transmitted to the beam splitter 230.

A third lens 250 is located between the beam splitter 230 and a detector 270, and a second light filter 260 is disposed between the third lens 250 and the detector 270. The second detector 260 is configured to ensure the needed Raman shifted peak intensity of the light source after scattering enters the detector 270. The detection light 214 passing through the second lens 240 is transmitted to the detector 270 by passing through the beam splitter 230, the third lens 250, and the light filter 260. Since a distance from the third lens 250 to the detector 270 is substantially equal to a focal length of the third lens 250, the detection light 214 entering the third lens 250 in parallel can be focused at the detector 270 after exiting the third lens 250.

In the present embodiment, the detector 270 may be a photomultiplier tube (PMT), a charge coupled device (CCD), an avalanche photo diode (APD), or a complementary metal oxide semiconductor (CMOS) transistor, for example, although the type of the detector 270 is not limited thereto.

Moreover, although the present embodiment adopts a plurality of lenses and beam splitters to transmit light to the testing area and the detector, the quantity and the position of the lenses are not limited by the above description, so long as light can be transmitted to the testing area, and the detection light can be transmitted to the detector after passing through the testing area. In other embodiments, light can be transmitted by using optical fibers.

In view of the foregoing, by exciting the test substance to generate the Raman effect, the optical detection method according to some embodiments of the disclosure enhances the measurement for trace concentrations of the test substance. Since the measured signal is increased, a high power incident light is not required. Moreover, the optical detection method is a non-radioactive and label-free in vitro test that could achieve early and safe detection of Alzheimer's disease. Furthermore, when measuring the concentration of β-amyloid in the eye, the concentration of β-amyloid can be respectively measured for the aqueous humor, lens, and retina. Moreover, the accuracy of the diagnosis can be increased by evaluating the measured concentrations together.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A method for detecting a concentration of a substance in an eye, the method comprising:
selecting β-amyloid (Aβ) as the substance;
emitting a light to a testing area in the eye, wherein a frequency of the light is selected according to an absorption spectrum of the selected substance, and the frequency is equal or close to a resonant excitation frequency of one of the electronic molecular energy levels of the substance, so as to excite the substance to generate resonance-enhanced Raman effect or pre-resonance Raman effect to form a detection spectrum; and
receiving the detection spectrum and estimating the concentration of the substance according to a peak intensity of the detection spectrum.

2. The method as claimed in claim 1, wherein the testing area is an aqueous humor, a lens, or a retina.

3. The method as claimed in claim 1, wherein the light is emitted from a light source and is focused in front of the eye by passing through a first lens.

4. The method as claimed in claim 3, wherein the light source is a light emitting diode or a laser diode.

5. The method as claimed in claim 1, wherein the light is transmitted to the testing area of the eye by passing through a beam splitter, and a detection light generated by the light passing through the testing area is transmitted to a detector by passing through the beam splitter.

6. The method as claimed in claim 5, wherein a second lens is disposed between the testing area and the beam splitter, and a distance from the testing area to the second lens is substantially equal to a focal length of the second lens.

7. The method as claimed in claim 5, wherein a third lens is disposed between the beam splitter and the detector, and a distance from the third lens to the detector is substantially equal to a focal length of the third lens.

8. The method as claimed in claim 5, wherein the detector is a photomultiplier tube (PMT), a charge coupled device (CCD), an avalanche photo diode (APD), or a complementary metal oxide semiconductor (CMOS) transistor.

9. The method as claimed in claim 1, wherein the β-amyloid is β-amyloid (1-40) or β-amyloid (1-42).

10. The method as claimed in claim 1, wherein a wavelength range of the light is from 300 nm to 330 nm.

11. A method for detecting a concentration of β-amyloid in a lens of an eye, the method comprising:
emitting a light of a frequency to the lens, wherein the frequency of the light is equal or close to a resonant excitation frequency of one of the electronic molecular energy levels of a deposit of a combination of αβ-crystallin and β-amyloid, so as to excite the deposit of the combination of αβ-crystallin and β-amyloid to generate resonance-enhanced Raman effect or pre-resonance Raman effect to form a detection spectrum; and
receiving the detection spectrum and estimating the concentration of the deposit of the combination of αβ-crystallin and β-amyloid according to a peak intensity of the detection spectrum, so as to obtain the concentration of β-amyloid in the lens.

12. The method as claimed in claim 11, wherein the light is emitted from a light source and is focused in front of the eye by passing through a first lens.

13. The method as claimed in claim 12, wherein the light source is a light emitting diode or a laser diode.

14. The method as claimed in claim 11, wherein the light is transmitted to the lens by passing through a beam splitter, and a detection light generated by the light passing through the lens is transmitted to a detector by passing through the beam splitter.

15. The method as claimed in claim 14, wherein a second lens is disposed between the lens and the beam splitter, and a distance from the lens to the second lens is substantially equal to a focal length of the second lens.

16. The method as claimed in claim 14, wherein a third lens is disposed between the beam splitter and the detector, and a distance from the third lens to the detector is substantially equal to a focal length of the third lens.

17. The method as claimed in claim 14, wherein the light source is a PMT, a CCD, an APD, or a CMOS transistor.

18. The method as claimed in claim 11, wherein a wavelength range of the light is from 440 nm to 460 nm.

19. A method for detecting a concentration of β-amyloid (Aβ) in an object, the method comprising:
emitting a light to a testing area, wherein a frequency of the light is selected according to an absorption spectrum of β-amyloid (Aβ), and the frequency is equal or close to a resonant excitation frequency of one of the electronic molecular energy levels of β-amyloid (Aβ), so as to excite β-amyloid (Aβ) to generate resonance-enhanced Raman effect or pre-resonance Raman effect to form a detection spectrum; and
receiving the detection spectrum and estimating the concentration of β-amyloid in the object according to a peak intensity of the detection spectrum.
